(19) United States
(12) Patent Application Publication
(10) Pub. No.: US 2002/0156151 A1
(43) Pub. Date: Oct. 24, 2002

(54) Water Soluble polymers containing cyclic amide or vinyl amide residues for dental restoratives

(76) Inventors: Bill M. Culbertson, Columbus, OH (US); Minhoa H. Dotrong, Dublin, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/040,888

(22) Filed: Jan. 7, 2002

Related U.S. Application Data
(60) Provisional application No. 60/259,934, filed on Jan. 5, 2001.

(51) Int. Cl. C08F 20/58; A61F 2/00

(52) U.S. Cl. 523/115; 526/304

(57) ABSTRACT

Improved polymers for use in dental restoratives are provided, as well as dental restoratives made with these improved polymers. The polymers of the present invention are made from at least one carboxylic acid-containing monomer and at least one vinyl amide monomer, and have molecular weights in the range from about 10,000 to about 100,000. In one embodiment, the polymer is made from two different acid monomers and one vinyl amide monomer. The present invention also provides for carboxylic acid and amine-containing polymers that are made visible light curable through the addition of a pendant vinyl-containing group. The invention also relates to methods of making the improved polymers and methods of making dental restoratives with the improved polymers. The invention also relates to kits for making dental restoratives comprising the carboxylic acid and amine-containing polymers of the present invention and an inorganic glass which are blended to form the dental restorative.

### CROSS-REFERENCE TO RELATED APPLICATION

This application claims priority to U.S. Provisional Application 60/259,934, filed Jan. 5, 2001, which is incorporated herein in its entirety.

This invention was made, at least in part, with government support under National Institutes of Health Grant No: DE 11682-03 awarded by the National Institute of Dental and Craniofacial Research. The U.S. government has certain rights in the invention.

### BACKGROUND OF THE INVENTION

Dental restoratives, or cements, have many applications in dentistry, such as filling materials for restoring teeth, cementing inlays and crowns into teeth, providing a base or lining in a tooth cavity, providing a temporary fixing for the bonds of orthodontic appliances to teeth, or sealing root-canals after endodontic treatment.

One important type of modem dental restorative consists of a water-soluble, organic polymer system blended with an inorganic filler, usually a glass powder. The organic polymers used in these restoratives normally contain carboxylic acid groups. These water-soluble organic polymers and co-polymers can be formed, for example, from acrylic acid and maleic acid. Such polymers are commercially available or, alternatively, can be made in the laboratory by mixing the carboxylic acid-containing monomers in an aqueous solution containing alcohol and initiating the reaction by heating the mixture to a temperature of about 95° C. in the presence of added free-radical initiators, such as potassium persulfate.

To obtain the functional restorative, the organic polymer is blended with an inorganic glass, such as for example, calcium fluoroaluminosilicate (CaFAlSiO₂) glass. The calcium and aluminum cations in the glass powder react with the ionized carboxylic acid groups of the polymer, in what is essentially an ionic acid-base neutralization reaction, to form metal polysalt or salt-bridges. Salt-bridge formation results in production of a hardened, insoluble matrix.

Dental restoratives of the type described above, i.e., glass poly(alkeonates) or more commonly called glass-ionomers (GI), are widely used in dentistry because of advantages over traditional mercury-containing amalgams. Such advantages include superior esthetics, little to no shrinkage on curing, better biocompatibility, release of fluoride ion, an attractive coefficient of thermal expansion, good adhesion to tooth structure, and lack of mercury.

In spite of these advantages in the current glass-ionomer dental restoratives, a need still exists for dental restoratives with greater mechanical durability. Especially desirable are new dental restoratives with increased strength, increased resistance to wear, and increased ability to withstand biting forces under in vivo conditions.

### SUMMARY OF THE INVENTION

The present invention relates to new polymer compositions for use in glass-ionomer dental restoratives. Dental restoratives made with these new polymer compositions offer a greatly improved resistance to bending and resistance to twisting over presently available glass-ionomer dental restoratives. Furthermore, the dental restoratives made in accordance with the present invention offer as good or better resistance to compression as the present restoratives.

The present invention relates to new polymer compositions for use in dental restoratives. These polymers are formed from at least two different monomers, one monomer containing a vinyl group and at least one carboxylic acid group, and the second monomer containing a vinyl amide. The vinyl amide may, but does not have to be cyclic. The vinyl amide that is used is one that is free-radical polymerizable. Some examples of cyclic vinyl amide monomers that can be used with the present invention include N-vinyl-2-pyrrolidone, N-vinylcarbazole, N-vinylsuccinimide, N-vinylcaprolactam, and N-vinylimidazole. Examples of non-cyclic vinyl amide monomers that can be used in the present invention include acrylamide, methacrylamide, dimethylacrylamide or isopropylacrylamide. Any of several monomers can be used for the vinyl and carboxylic acid-containing monomer. Examples of these include: acrylic acid, methacrylic acid, itaconic acid, N-acryloyl and N-methacryloylamino acids, maleic acid and citraconic acid.

In accordance with the present invention it may be desirable to use two or more different acid monomers, along with the vinyl amide monomer to form the polymer. Furthermore, in accordance with the present invention, a free-radical polymerizable or visible light curable group can be added to the polymer to enhance its utility as a dental restorative. Such groups include 2-isocyanatoethyl methacrylate and glycidyl methacrylate, as well as acrylates, methacrylates, acrylamides, and methacrylamides added via oxazine or oxazoline ring-opening reactions.

The present invention also relates to dental restoratives made with the inventive polymer. Glass-ionomer dental restoratives are formed by the addition of a suitable inorganic glass powder, generally an calcium aluminum fluorosilicate glass, to the polymer. In accordance with the present invention, the polymer containing vinyl amide groups and carboxylic acid groups is blended with the inorganic glass powder, the resulting mixture is applied to a dental area in need of restoration, the restorative is then cured in situ.

The present invention also relates to methods of making polymers from monomers containing a vinyl group and at least one carboxylic acid group and monomers containing a vinyl amide. The present invention also relates to methods of adding free-radical or visible light curable groups to these polymers. The present invention also relates to methods of making dental restoratives using these polymers.

The present invention also relates to kits for making dental restoratives in accordance with the present invention. The kits will contain either a polymer solution or polymer precursors which can be reacted to form the polymer to be used in the dental restoratives. The kit may also include an inorganic glass for use in the dental restorative, as well as a free-radical or visible light curing agent. Other desirable additives that may be added to the dental restorative mixture, for example to enhance wear or esthetics, may be included in the kit as well. Instructions for preparation and use of the dental restoratives will be another part of the kit.

### DETAILED DESCRIPTION OF THE INVENTION

The present invention provides methods for making organic polymers containing vinyl amides. These resulting polymers form the basis of the improved dental restoratives of the present invention. Such polymers are made by including vinyl amide monomers along with the carboxylic acid-containing monomers in the polymerization reaction mixture. The vinyl amide is generally used at a concentration of about 25 mole percent or less. More preferably, the vinyl amide is used at a concentration of about 5-10 mole percent.

The carboxylic acid-containing vinyl monomers used to form the polymer include, but are not limited to, acrylic acid, methacrylic acid, itaconic acid, N-acryloyl and N-methacryloylamino acids, maleic acid and citraconic acid. In accordance with the present invention, it may be desirable to use combinations of two or more different carboxylic-acid containing vinyl monomers along with the vinyl amide monomer. Suitable vinyl amide monomers include, but are not limited to N-vinyl-2-pyrrolidone, N-vinylcarbazole, N-vinylsuccinimide, N-vinylcaprolactam, N-vinylimidazole, acrylamide, methacrylamide, dimethylacrylamide, and isopropylacrylamide.

In one embodiment, acrylic acid (AA), maleic acid (MA) and NVP are dissolved together in an aqueous polymerization solution, along with a suitable amount of potassium persulfate initiator, in a molar ratio of 7:3:1, respectively. After reacting the components at 95° C. for 24 hours, the poly(AA-co-MA-co-NVP) terpolymer, with a 7:3:1 monomers ratio, is collected, purified and characterized. Such terpolymers can be made to have molecular weights in the range of 10,000-100,000. A general structure for such a terpolymer, (with x=7, y=3 and z=1,) is shown below.

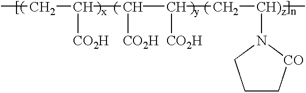

The polymer poly(AA-co-NVP) or, alternatively, the polymer poly(MA-co-NVP) could be synthesized in a manner similar to the description given above.

In another embodiment, the polymer is made with a vinyl amide other than N-vinyl-2-pyrrolidone. The general chemical structure for such a vinyl amide that could be used in the polymerization reaction is given below.

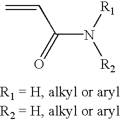

As noted in the structure, the nitrogen atom of the vinyl amide can be substituted with hydrogen, alkyl, and aryl groups. As used throughout the specification, the term “alkyl” embraces linear or branched radicals having one to about twenty carbon atoms. More preferred alkyl radicals are “lower alkyl” radicals having one to about ten carbon atoms. Even more preferred in the present invention are methyl and ethyl radicals. Examples of alkyl radicals include, but are not limited to, methyl, ethyl, n-propyl, isopropyl, n-butyl, isobutyl, sec-butyl, tert-butyl, pentyl, hexyl, octyl, and the like. The term “aryl” embraces aromatic radicals such as phenyl, naphthyl, tetrahydronaphthyl, indane, phenanthryl, anthracenyl, and biphenyl, as well as aryl groups that are substituted at any substitutable positions and arylalkyl and alkylaryl groups. It would also be recognized by those of skill in the art that further substitutions, such as with a halogen or lower alkyl, could be made at various substitutable positions on the substituents themselves, and still fall within the scope of the present invention.

The polymers of the present invention, having vinyl amides and carboxylic acid groups can be used, in accordance with the present invention, to make visible light-curable (VLC) polymers. Visible light curable polymers are formed by incorporating visible light curable residues into the polymer; these residues are incorporated in such a way that they are essentially tethered to, or pendant to, the polymer backbone. The polymer is then cured when an outside stimulus, such as a visible light source or other free radical polymerization initiator, is applied. In this way, the curing of the polymer can be precisely controlled. Acrylate, methacrylate, acrylamide and methacrylamide are especially useful residues for visible light curing. Two molecules that are well known in the art as useful for attaching these residues are 2-isocyanatoethyl methacrylate (IEM) and glycidyl methacrylate (GM).

Another recently introduced way of making visible light curable polymers is by adding the desired residue to the polymer through a ring opening reaction of a cyclic imino ether, as described in WO 011/53358 A1, published Jul. 26, 2001. Cyclic imino ethers, such as 1,3-oxazines and oxazolines, can be functionalized with the desired residue at the 2-position. The cyclic imino ether undergoes a ring-opening reaction and attaches to the polymer backbone. The desired residue is then effectively tethered to the polymer backbone. The use of cyclic imino ethers to add the desired residue is especially advantageous because the reaction can be carried out in aqueous solution. Examples of suitable functionalized cyclic imino ethers include 1,3-oxazines and oxazolines that have an acrylate, methacrylate, acrylamide, or methacrylamide group substituted at the 2-position. While these represent exemplary molecules for making visible light curable polymers, it is recognized that there are other suitable molecules known to those skilled in the art which could be incorporated into the polymer of the present invention without altering the scope of the invention.

Accordingly, in another embodiment, an NVP-containing polymer and a vinyl substituted cyclic imino ether are dissolved at a molar ratio of 1:5 (based on carboxylic acid groups), respectively, in an aqueous solution containing an alcohol. The solution is then heated to a temperature of about 65° C. for 4 hrs. The visible light curable NVP-containing polymer is then recovered from solution, purified and characterized. The general structure of such a visible light curable NVP-containing polymer is given below in structure A. The R group in structure A is defined below in structure B for different embodiments of the polymer.

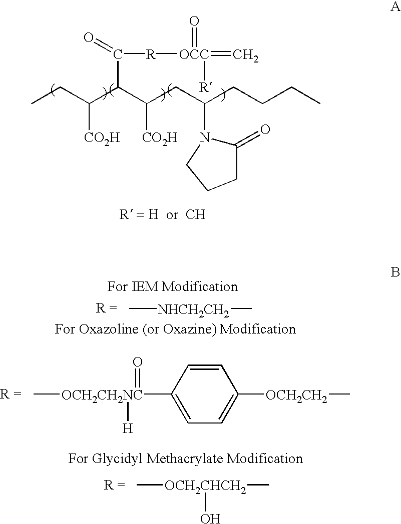

Vinyl amide-containing organic polymers, whether visible light curable or not, can be blended and reacted with CaFAlSi-type glass powders to make glass-ionomer type dental restoratives. Such glass-ionomer dental restoratives have improved strength and resistance to wear compared to glass-ionomer dental restoratives made with organic polymers that do not contain the vinyl amide.

In one embodiment, an NVP-containing polymer (50% aqueous solution) is combined with calcium fluoroaluminosilicate glass powder to give a mixture that is between 50-80% glass, by weight. The resulting glass ionomer dental restorative, which is a homogeneous, paste-like mixture, is applied to the dental area in need of restoration and allowed to cure, i.e., harden by the acid-base reaction in the mixture.

In another embodiment, a visible light curable NVP-containing polymer is blended with calcium fluoroaluminosilicate glass filler as above. Due to the presence of tethered methacrylate residues on the visible light curable NVP-containing polymer, a strong visible-light source can be used to catalyze formation of covalent bonds via a free-radical reaction. In such case, hardening or curing occurs both by cross-linking of the organic polymer matrix and the acid-base reaction of the carboxylate (CO₂⁻) anions and the Ca⁺⁺ or Al⁺⁺⁺ cations from the glass.

### EXAMPLES

### Example 1

### General Procedure for Preparing Vinyl Amide-Containing Polymers

A distilled water (260 mL)/potassium persulfate (0.94 g) initiator mixture was stirred under nitrogen for 30 min to remove any dissolved oxygen. Solutions of acrylic acid (65.58 g, 0.91 mol), maleic acid (45.27 g, 0.39 mol) and N-vinylpyrrolidone (0.94 g) in 80 mL of distilled water, were subdivided into 20 equal portions, with each portion of the two solutions added every five minutes to the polymerization reactor, which was kept at 95° C. The polymerization reaction was run for 24 hours at 95° C. The terpolymer was isolated by standard freeze drying techniques in which water vapor is removed under vacuum, leaving only the solid polymer residue. The solid terpolymer was dissolved in pure methanol. The methanol solution was then combined with a large excess of ethyl acetate which precipitated the polymer.

This polymer, designated “F” in Table 1 below, was made first and collected in a purified yield of greater than 76%. The structure of polymer F was confirmed both by FT-IR and NMR (¹H and ¹³C) spectroscopy. The spectra confirmed that acrylic acid, maleic acid and NVP were incorporated into the terpolymer backbone. Thereafter, the A-E polymers were made similarly, only varying the ratios of acrylic acid (AA), maleic acid (MA) and NVP, as shown in Table 1 below.

The table shows that some of the polymers have the same molar ratio of acrylic acid, maleic acid and NVP (polymers A and B are both 5:5:1; polymers D, E and F are all 7:3:1). In these cases, even though the molar ratios are identical, different amounts of potassium persulfate initiator were used in each formulation, resulting in different molecular weights for each polymer (as reflected in the viscosity measurements). Therefore, even though the molar ratio of reactants for some of the formulations were identical, all polymers were different, based on the molecular weight of each polymer.

TABLE 1
Composition and Viscosity of Polymers

| | Molar ratio | Viscosity (SD) | Yield stress (SD) |
|---|---|---|---|
| System | AA:MA:NVP | cP | Newtons/m² |
| Fuji II* | — | 562 (21) | 44 (17) |
| Fuji IX* | — | 565 (7) | 43 (11) |
| A | 5:5:1 | 730 (51) | 35 (22) |
| B | 5:5:1 | 1820 (5) | 63 (11) |
| C | 7:1:3 | 526 (52) | 12 (11) |
| D | 7:3:1 | 1946 (42) | 51 (39) |
| F | 7:3:1 | 1732 (38) | 103 (25) |
| F | 7:3:1 | 1109 (56) | 34 (21) |

These studies showed that it is possible to synthesize poly(acrylic acid-co-maleic acid-co-NVP) polymers in a wide variety of monomer ratios. These studies also show that addition of NVP increases, to some degree, the viscosity of the aqueous polymer solutions, helping to limit the amount of NVP which may be used to modify the AA-MA polymer. Further, NVP also reduces the amount of carboxylic acid groups present in the polymer backbone, which could, to some degree, cut down on the level of salt-bridge formation. However, NVP improves the overall compressive strength, flexural strength and toughness of the formulated glass-ionomer, when the NVP is used at a controlled, low level (i.e., concentrations less than or equal to 25 mole %).

### Example 2

### General Procedure for Preparing Visible Light Curable Vinyl Amide -Containing Polymers

To 0.050 mols (based on acrylic acid) of the purified terpolymer of Example 1 in 15 mL of water, 15 mL of isopropanol was added and the solution was warmed to 65° C. To the heated solution, 0.100 moles of methacrylated oxazoline was added and the reaction flask was fitted with a condenser. The reaction mixture was homogeneous and was allowed to stir for 4 hrs. The polymer was isolated from the reaction mixture by precipitation in water, purified and characterized with FT-IR and NMR, confirming the polymer contained the required methacrylate residues.

### Example 3

### Preparation and Characteristics of Restoratives Made with NVP-Containing Polymers and Fuji II or Fuji IX Glass Powder

NVP-containing polymers A-F were prepared and purified as described in Example 1 above. These polymers were then blended with the glass powder component of the commercially-available Fuji II GI kit (GC Corporation), using a 50% solution of polymer blended with glass powder at a ratio of 2.7:1 by weight in all cases. The Fuji II GI restorative (Fuji II aqueous polymer solution plus glass powder) was used as control. The polymer of the Fuji II kit is made from monomers of acrylic acid and maleic acid. The Fuji II polymer does not contain NVP.

Both for the A-F restoratives as well as the Fuji II GI restorative, mixing of the polymer solution with glass powder was performed with mixing times under 45 sec before placing the mixtures in molds. All samples were cured for 24 hr at 37° C. and 100% relative humidity. The test samples were then machined to certain sizes for compressive strength (CS; a measure of resistance to compression), flexural strength (FS; a measure of resistance to bending) and diametrial tensile strength (DTS; a measure of resistance to twisting) testing. Mechanical properties such as CS, FS and DTS are important properties for restorative dental materials since the restorative materials must withstand biting forces under in vivo conditions. CS and DTS testing were done using an Instron 4204 machine with a crosshead speed of 1 mm/min and a load cell of 1000 kg. For FS testing, the crosshead speed was as above with a load cell of 100 kg. Sample size for CS testing was an 8 mm×4 mm cylinder. FS testing required small bars having 25 mm×2 mm×2 mm dimensions. DTS testing used a disc size of 4 mm diameter and 2 mm thick. The values obtained for testing of these restoratives are shown in Table 2.

TABLE 2
Mechanical Properties of the Polymers Formulated with Fuji II Glass Powder

| | Compressive | Compressive | Flexural | Flexural | Diametral tensile |
|---|---|---|---|---|---|
| | strength (SD) | modulus (SD) | strength (SD) | modulus (SD) | strength (SD) |
| System | MPa | GPa | MPa | GPa | MPa |
| Fuji II | 248.6 (5.9) | 6.97 (0.11) | 11.92 (1.34) | 5.11 (0.93) | 13.30 (2.20) |
| A | 255.6 (16.1) | 7.63 (0.13) | 35.50 (7.59) | 14.01 (0.75) | 16.11 (1.21) |
| B | 205.0 (18.5) | 7.50 (0.18) | 43.98 (2.12) | 17.06 (0.35) | 15.37 (1.35) |
| C | 188.0 (12.9) | 5.48 (0.19) | 36.95 (4.87) | 14.63 (0.36) | 15.76 (1.42) |
| D | 241.9 (9.8) | 6.45 (0.28) | 39.84 (4.76) | 17.80 (0.62) | 19.63 (1.67) |
| E | 216.5 (17.9) | 5.80 (0.28) | 41.64 (4.05) | 15.55 (0.91) | 18.99 (0.78) |
| F | 219.7 (19.9) | 6.04 (0.27) | 38.94 (4.68) | 16.87 (0.43) | 17.92 (5.19) |

In addition to blending NVP-containing polymers with the glass powder from the Fuji II GI kit, NVP-containing polymers A-F were also blended with the glass powder component of the commercially-available Fuji IX GI kit. The Fuji IX GI restorative (Fuji aqueous polymer solution plus glass powder) was also used as a control. The polymer of the Fuji IX kit is made from monomers of acrylic acid and maleic acid. The Fuji IX polymer does not contain NVP. Both the restoratives containing the A-F polymers, as well as the Fuji IX commercial restorative were blended, formed and mechanically tested, as described above. The values obtained for testing of these restoratives are shown in Table 3.

TABLE 3
Mechanical Properties of the Polymers Formulated with Fuji IX Glass Powder

| | Compressive | Compressive | Flexural | Flexural | Diametral tensile |
|---|---|---|---|---|---|
| | strength (SD) | modulus (SD) | strength (SD) | modulus (SD) | strength (SD) |
| System | MPa | GPa | MPa | GPa | MPa |
| Fuji IX | 281.1 (18.7) | 6.82 (0.23) | 16.65 (1.88) | 16.45 (0.86) | 16.99 (2.02) |
| A | 289.6 (21.3) | 6.60 (0.19) | 40.48 (4.62) | 21.24 (0.62) | 18.23 (1.14) |
| B | 267.1 (12.8) | 6.64 (0.12) | 48.55 (3.50) | 20.20 (0.41) | 17.27 (1.72) |
| C | 185.9 (22.6) | 5.89 (0.41) | 35.92 (5.90) | 16.49 (0.62) | 15.95 (1.91) |
| D | 307.0 (28.0) | 6.80 (0.29) | 30.08 (3.35) | 20.10 (1.19) | 21.02 (2.44) |
| E | 265.0 (22.1) | 6.69 (0.43) | 39.38 (3.44) | 16.49 (1.42) | 18.24 (1.65) |
| F | 231.3 (16.3) | 6.66 (0.27) | 32.07 (5.43) | 20.58 (1.36) | 19.99 (1.49) |

The results of these studies (Tables 2 and 3) show that GI restoratives made with NVP-containing polymers have significantly increased flexural strength (FS) as compared to control GI restoratives made without NVP. FS is related to GI toughness, which is an important characteristic for optimal use of GI restoratives. Therefore, in this respect, the GI restoratives made with the NVP-containing polymers are improved as compared to the control restoratives made with polymers that do not contain NVP.

The results of these studies also show that the improvement in FS shown by the restoratives made with the NVP-containing polymers does not cause a decrease in compressive strength (CS) or in diametral tensile strength (DTS). The CS and DTS values obtained for all restoratives made with the NVP-containing polymers are within the range required for these restoratives to function well.

What is claimed is:

1. A polymer for use in dental restoratives wherein the polymer has a backbone structure comprising:
a) a first monomer unit comprising a carboxylic acid-functionalized vinyl monomer; and
b) a second monomer unit comprising a vinyl amide.

2. The polymer of claim 1, wherein the polymer further comprises a free-radical or visible light curable moiety pendant to the polymer backbone.

3. The polymer of claim 2 wherein said free-radical or visible light curable moiety is selected from the group consisting of vinyl-substituted unsaturated cyclic imino ethers, 2-isocyanatoethyl methacrylate, and glycidyl methacrylate.

4. The polymer of claim 1 wherein said carboxylic acid-functionalized vinyl monomer is selected from the group consisting of acrylic acid, maleic acid, itaconic acid, methacrylic acid, citraconic acid, N-acryloyl substituted amino acids, N-methacryloyl substituted amino acids, and combinations thereof.

5. The polymer of claim 4, wherein said polymer comprises at least two of said carboxylic acid-functionalized vinyl monomers.

6. The polymer of claim 1 wherein the vinyl amide is selected from the group consisting of acrylamide, methacrylamide, dimethylacrylamide, isopropylacrylamide, N-vinyl-2-pyrrolidone, N-vinylcarbazole, N-vinylsuccinimide, N-vinylcaprolactam, and N-vinylimidazole.

7. The polymer of claim 6 wherein the vinyl amide is N-vinyl-2-pyrrolidone.

8. The polymer of claim 1 wherein the concentration of the vinyl amide ranges from about 5 to about 25 mole percent.

9. The polymer of claim 8 wherein the concentration of the vinyl amide ranges from about 5 to about 10 mole percent.

10. A polymer for use in dental restoratives wherein the polymer has a backbone structure comprising:
a) a first monomer unit comprising a carboxylic acid-fanctionalized vinyl monomer; and
b) a second monomer unit comprising a free-radical polymerizable amide.

11. A dental restorative comprising:
a) the polymer of claim 1; and
b) an inorganic glass powder;
wherein the dental restorative is formed when said polymer is blended with said inorganic glass powder.

12. The dental restorative of claim 11, wherein the polymer has a molecular weight in the range of about 10,000 to about 100,000.

13. The dental restorative of claim 11 wherein the inorganic glass powder is a calcium fluoroaluminosilicate glass.

14. The dental restorative of claim 11 further comprising a free-radical or visible light curable moiety pendant to said polymer, said free-radical or visible light curable moiety selected from the group consisting of vinyl-substituted unsaturated cyclic imino ethers, 2-isocyanatoethyl methacrylate, and glycidyl methacrylate.

15. A method for preparing a polymer to be used in dental restoratives comprising:
a) polymerizing monomers comprising:
i) carbon chains containing a carboxylic acid group at one or both ends and containing at least one carbon-carbon double bond; and
ii) a vinyl amide; and
b) recovering said polymer from the reaction mixture.

16. The method of claim 15, wherein the recovered polymer is reacted with vinyl-substituted unsaturated cyclic imino ethers, 2-isocyanatoethyl methacrylate, or glycidyl methacrylate, to produce a visible light-curable polymer.

17. A free-radical or visible light curable dental restorative comprising:
a) the polymer of claim 16; and
b) an inorganic glass powder.

18. A method for preparing a dental restorative comprising:
a) blending reactants comprising:
i) a polymer formed from at least one carboxylic acid-containing vinyl monomer and at least one vinyl amide monomer;
ii) an inorganic glass powder; and
b) applying the blended reactants to a dental area in need of restoration; and
c) curing the applied blended reactants.

19. A kit for use in making dental restoratives comprising:
a) a polymer comprising carboxylic acid groups and amide groups; and
b) an inorganic glass powder;
wherein the dental restorative is made by blending said polymer with said inorganic glass powder.

20. A kit for use in making dental restoratives comprising:
a) a carboxylic acid-containing vinyl monomer;
b) a free-radical polymerizable amide-containing monomer; and
c) an inorganic glass powder;
wherein a polymer is formed from said carboxylic acid-containing vinyl monomer and said free-radical polymerizable amide and said inorganic glass powder is blended with the polymer to form the dental restorative.
